(19) United States
(12) Patent Application Publication
(10) Pub. No.: US 2012/0130269 A1
(43) Pub. Date: May 24, 2012

(54) INTEGRATED NERVE STIMULATION AND SKIN MARKING DEVICE AND METHODS OF USING SAME

(75) Inventor: Ryan M. Rea, Ventura, CA (US)

(73) Assignee: NEURAL PATHWAYS, LLC, Ventura, CA (US)

(21) Appl. No.: 13/290,651

(22) Filed: Nov. 7, 2011

Related U.S. Application Data
(60) Provisional application No. 61/415,553, filed on Nov. 19, 2010.

(51) Int. Cl. A61B 5/05; A61B 17/3209; A61N 1/36; A61B 18/14; A61N 1/34

(52) U.S. Cl. 600/554; 606/41; 607/46; 607/42; 606/167

(57) ABSTRACT

A nerve identification device is shown and described. The device includes both a nerve stimulating electrode and a skin marking device. In certain applications, a user contacts the patient's skin with the nerve stimulating electrode to deliver nerve stimulation energy that evokes an electrical response from proximally located motor nerves. When nerves are detected, the user applies skin ink via the device to the user's skin to identify the location where nerves were found for subsequent use in a variety of therapeutic procedures such as surgical procedures, pain treatment procedures, degenerative nerve destruction procedures, and the treatment of obstructive sleep apnea.

### CROSS-REFERENCE TO RELATED APPLICATIONS

This application claims the benefit of U.S. Provisional Patent Application No. 61/415,553, filed Nov. 19, 2010, the entirety of which is hereby incorporated by reference.

### FIELD

The present disclosure relates to nerve identification devices, and more specifically, to devices having both nerve stimulation and skin marking features.

### BACKGROUND

Electromyography (hereafter “EMG”) is used in Intraoperative Neurophysiological Monitoring (hereafter “IONM”) as a way to electromechanically detect “at risk” human motor nerves during surgery. Most human muscles contain human motor nerves. When human motor nerves are electrically stimulated or “excited” the muscles that contain those nerves contract. The muscle contractions are an evoked electromyographic (EMG) event commonly known as a Compound Action Potential (hereafter “CAP”) amongst the IONM community. Traditionally, EMG technologies have been incorporated into IONM hardware “systems” and accompanying disposable product accessories, such as disposable EMG recording electrodes, to detect at risk motor nerves in surgery. In operation, an IONM system generates a CAP by the surgeon evoking nerve responses by using electronically activated manual surgical instruments that stimulate at risk motor nerves and subsequently cause the muscles that contain them to contract. The electronically evoked nerve responses' subsequent muscle contractions are then recorded by EMG recording electrodes and transmitted to an IONM system's generator, which in turn, provides an audio and visual warning indication to an at risk nerve(s) to the surgeon and/or attending IONM clinician.

Certain percutaneous electrical nerve stimulation or “PENS” techniques have also been used. In such known PENS methods, a stimulation electrode in the form of a needle is inserted through the patient's skin to a location of interest, and stimulating energy is supplied. In PENS procedures, it is desirable to identify locations on a patient's skin where stimulation energy produces an evoked response, such as a CAP. Skin marking devices are available and can be used to mark the identified locations in such applications. However, known skin marking devices are separate from the nerve stimulating device and often require the surgeon to put down the nerve stimulation device and pick up the skin marking device. The exchange of devices can lead to inaccuracies in marking the correct location at which an evoked response was obtained and also tends to lengthen PENS procedures. In addition, known PENS procedures require the insertion of needle electrodes through the patient's skin, which can be painful.

### BRIEF DESCRIPTION OF THE DRAWINGS

The disclosure will now be described, by way of example, with reference to the accompanying drawings, in which:

FIG. 1 is a depiction of a nerve monitoring system in accordance with the present disclosure;

FIG. 2A is a longitudinal cross-sectional view of a first nerve identification device comprising a nerve stimulation electrode and a skin marker;

FIG. 2B is a close-up view of a distal portion of the nerve identification device of FIG. 2A;

FIG. 2C is a longitudinal cross-sectional view of the proximal and middle handpiece sections of the nerve identification device of FIG. 2A;

FIG. 2D is a longitudinal cross-sectional view of the distal handpiece section of the nerve identification device of FIG. 2A;

FIG. 2E is a longitudinal cross-sectional view of the conductive shaft of the nerve identification device of FIG. 2A;

FIG. 2F is a longitudinal cross-sectional view of the skin marking shaft of the nerve identification device of FIG. 2A;

FIG. 2G is a close-up of the distal tip of the nerve identification device of FIG. 2A showing the skin marker in a deployed (extended) condition;

FIG. 2H is a close-up of the distal tip of the nerve identification device of FIG. 2A showing the skin marker in an undeployed (retracted) condition;

FIG. 3A is a plan view of a second nerve identification device comprising a nerve stimulating electrode and a skin marker;

FIG. 3B is a close-up view of a distal portion of the nerve identification device of FIG. 3A;

FIG. 4 is an illustration of a surgeon using the skin marking device of FIG. 3A; and

FIG. 5 is a flow chart used to illustrate a method of identifying nerve locations on a patient's skin using auto-calibrated nerve stimulation energy levels.

### DETAILED DESCRIPTION

The present disclosure relates to a nerve identification device, and more specifically, to a nerve identification device comprising both a nerve stimulating electrode and a skin marker. The device allows a surgeon to supply nerve stimulation energy percutaneously (through the patient's skin) and to mark the skin upon the detection of selected evoked nerve responses, such as compound action potentials (CAPs), using a single, handheld device.

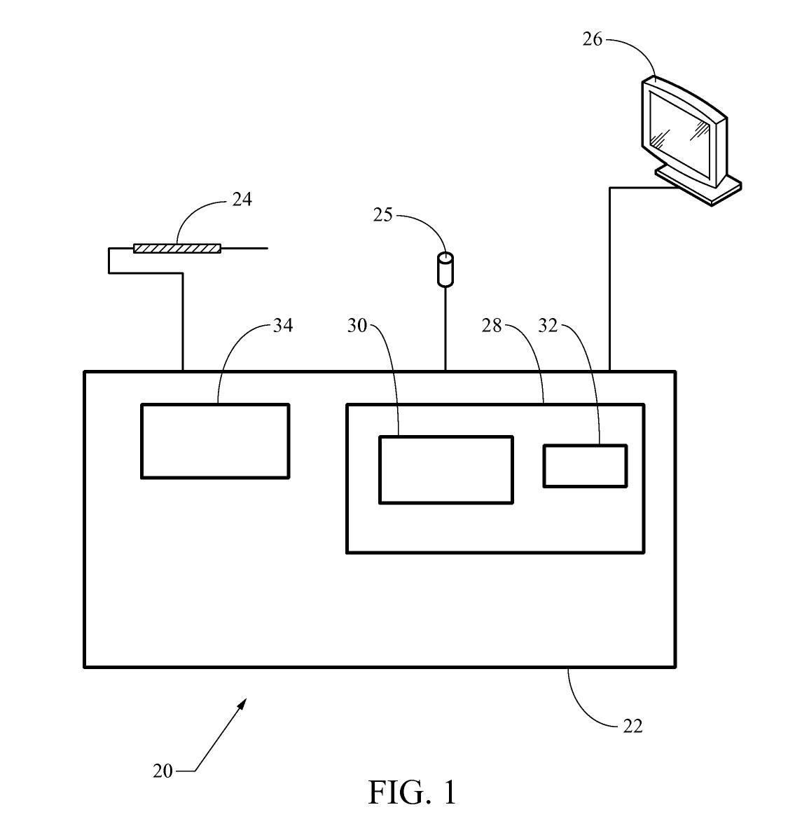

Referring to FIG. 1, a nerve monitoring system 20 is depicted. The nerve monitoring system 20 comprises a nerve identification device 24, an evoked nerve response sensor electrode 25, and a surgeon interface module 22. Surgeon interface module 22 is used to provide stimulation energy to a patient and to receive evoked nerve response signals from the patient. Surgeon interface module 22 comprises an electrical stimulation generator 34 and an evoked nerve response monitoring unit 28. The electrical stimulation generator 34 and evoked nerve response monitoring 28 unit may be provided in a single housing or may be separate from one another. Evoked nerve response monitoring unit 28 includes processor 30 and data storage 32 (e.g., hard disk drive, CD/DVD drive, etc.). In those implementations in which the electrical stimulation generator 34 and evoked nerve response monitoring unit 28 are enclosed in a common housing, the housing preferably has stimulation energy outputs which are electrically connectable to nerve identification device 24 and to electrical stimulation generator 34. The housing also preferably includes evoked nerve response sensor electrode inputs connected to evoked nerve response sensor electrode 25 and to evoked nerve response monitoring unit 28. System 20 may also include a user interface for adjusting the stimulation energy supplied to nerve identification device 24. Monitor 26 is also provided to allow the surgeon to receive visual indications regarding the generated stimulation energy and received evoked nerve response data generated by a patient's nerves.

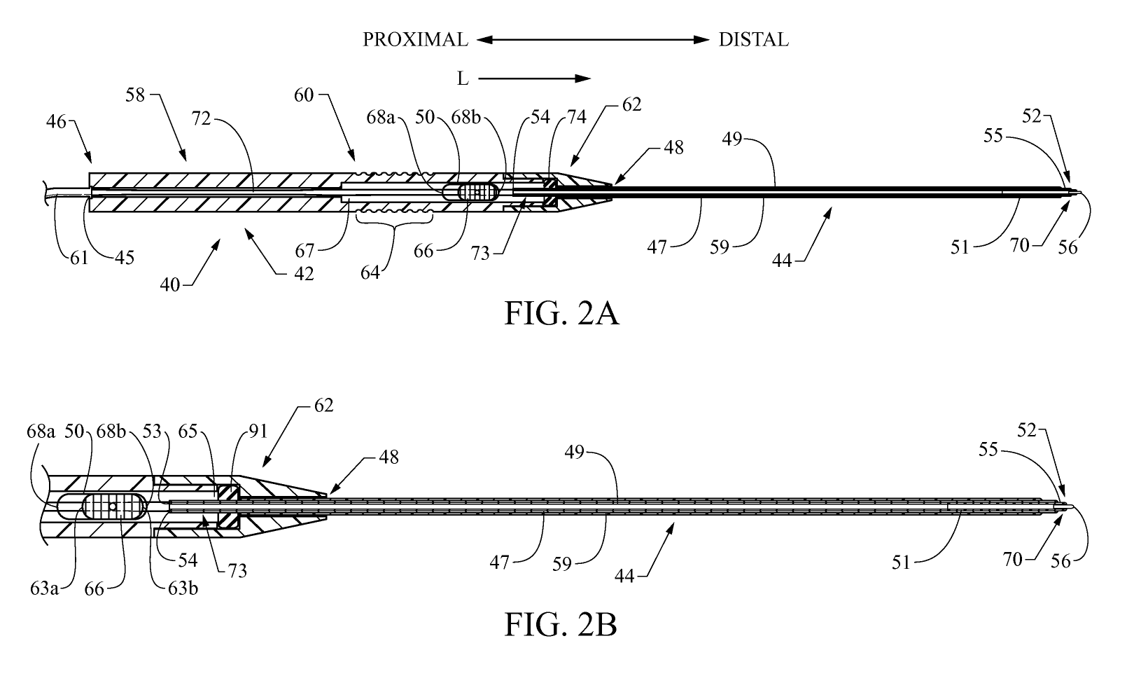

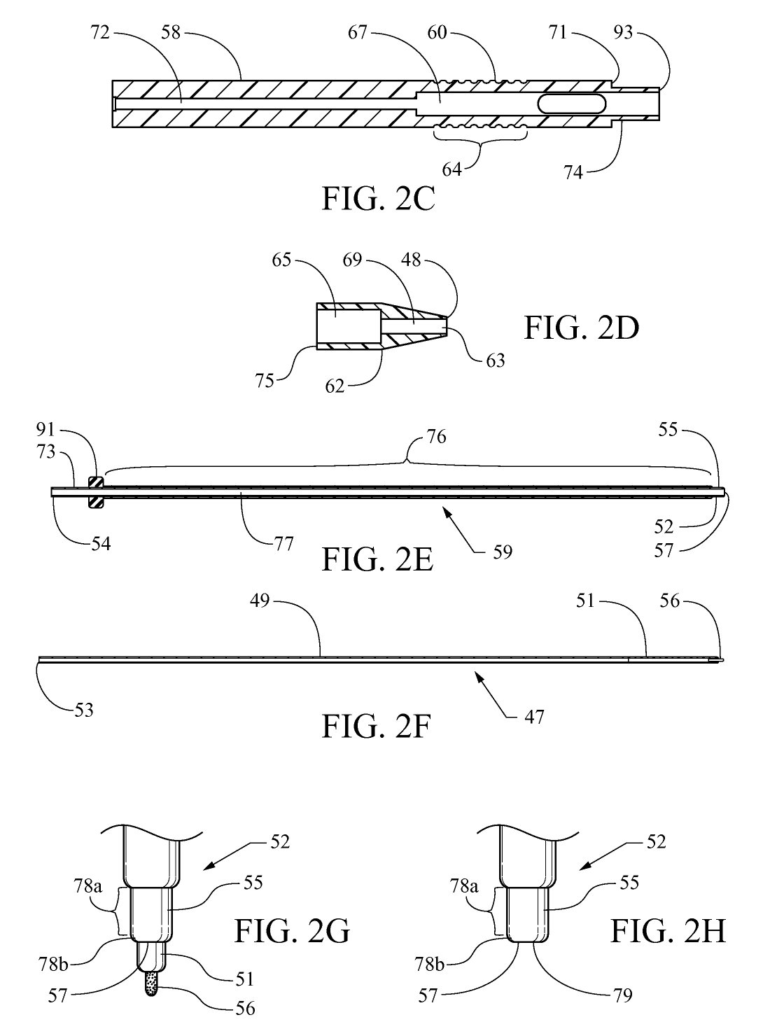

Referring to FIG. 2A, an exemplary nerve identification device 40 in accordance with the present disclosure is depicted. Nerve identification device 40 comprises a nerve stimulating electrode 55 and a skin marker 47 (FIG. 2F). Nerve identification device 40 also includes a handpiece 42 and a shaft section 44. Proximal end 46 of nerve identification device 40 is spaced apart from distal end 70 along the direction of the length L of device 40. Port 45 is provided at the proximal end 46 and receives an electrical conduit 61, which may include one or more conductive leads or wires for providing stimulating energy to nerve stimulating electrode 55. Nerve stimulating electrode 55 is disposed on the distal end of conductive shaft 59, thereby defining a conductive path for nerve stimulating energy to travel along the direction of device length L from electrical conduit 61 to conductive shaft 59, and to nerve stimulating electrode 55.

Handpiece 42 comprises proximal section 58, middle section 60, and distal section 62. Proximal section 58 includes a lumen 72 through which electrical conduit 61 is disposed. Proximal section lumen 72 is in communication with middle section lumen 67, through which electrical conduit 61 is also disposed. Distal section 62 also includes a proximal distal section lumen 65 and a distal distal section lumen 69 (FIG. 2D), which are in communication with one another and with middle section lumen 67. Proximal distal section lumen 65 is defined within a cylindrical proximal portion of distal handpiece section 62, and lumen 63 is defined within a distal tapered (frustoconical) portion of distal handpiece section 62. In the illustrated example, proximal distal section lumen 65 is located proximally from lumen 69 and has a larger cross-sectional area perpendicular to the device length L than does distal distal section lumen 69.

FIGS. 2C-2F depict handpiece 42 in an unassembled configuration. FIG. 2C depicts the proximal 58 and middle 60 sections of handpiece 42, while FIG. 2D depicts the distal section 62 of handpiece 42. Tactile features 64, such as ridges, are provided on the outer surface of middle section 60 to facilitate gripping with a single hand. Middle handpiece section 60 also includes a distal neck 74 which fits into proximal distal section lumen 65 of distal section 62. Distal annular face 71 is formed on middle section 60 of handpiece 42 and abuttingly engages proximal annular face 75 formed on distal handpiece section 62. The middle 60 and distal 62 sections may be joined by mechanical fasteners and/or adhesives. In addition, the sections may be configured for snap fitting engagement with one another. A sealing ring 91 is provided on conductive shaft 59 and engages distal face 93 of distal neck 74, as best seen in FIGS. 2B, 2C, and 2E.

Shaft section 44 comprises a conductive shaft 59 having an interior lumen 77 (FIG. 2E) within which skin marker 47 (FIG. 2F) is disposed. As best seen in FIG. 2E, conductive shaft 59 comprises a proximal end 54, an exposed proximal portion 73, an exteriorly insulated portion 76, and a distal end 52, which defines the distal end 70 of nerve identification device 40. Nerve stimulating electrode 55 is located at the conductive shaft distal end 52. Conductive shaft 59 is stationary relative to handpiece 42 and is formed from an electrically conductive material. Suitable conductive materials include medical grade steels, such as a medical grade SAE 303 stainless steel. Electrode 55 may be integrally formed with conductive shaft 59 or separately formed and attached thereto. In one exemplary implementation, electrode 55 is separately formed and welded onto conductive shaft 59. Nerve stimulating electrode 55 is also formed from an electrically conductive material, including medical grade steels such as a medical grade SAE 303 stainless steel.

In certain examples, shaft section 44 has a total length (as measured from proximal shaft end 54 to distal end shaft end 52) to outer diameter (i.e., based on the outer diameter of conductive shaft 59) ratio that ranges from 0.004 to 0.010, preferably from 0.005 to 0.007, and more preferably from 0.0055 to 0.0065. In other examples, shaft section 44 has a total length of from about 8 inches (20.3 cm) to about 14 inches (35.6 cm), preferably from about 11.5 inches (29.2 cm) to about 13.5 inches (34.3 cm) and more preferably from about 12.0 inches (30.5 cm) to about 13.0 inches (33.0 cm). In one example, the length of shaft section 44 is 12.5 inches (31.8 cm). In certain examples, shaft section 44 has an outer diameter of from about 0.06 (0.15 cm) to about 0.09 inches (0.23 cm), preferably from about 0.068 inches (0.172 cm) to about 0.078 inches (0.20 cm), and more preferably from about 0.070 inches (0.18 cm) to about 0.075 inches (0.10 cm). In one example, the outer diameter is 0.072 inches (0.18 cm).

Exteriorly insulated portion 76 of conductive shaft 59 comprises a portion of conductive shaft 59 having an electrically insulating material disposed on its exterior surface. The insulating material may be coated on the outer surface of the conductive shaft or may be separately formed and attached by an adhesive or other means such as heat shrinking. Suitable insulating materials include medical grade plastics such as those made from fluorinated polymers. Suitable fluorinated polymer plastics include heat shrinkable PTFE (poly tetrafluoroethylene, including Teflon®) or FEP (fluorinated ethylene-propylene). In one example, FEP heat shrink tubing is used.

Exposed proximal portion 73 of conductive shaft 59 has an exposed conductive exterior surface and provides a location for making electrical contact with electrical conduit 61. In certain examples, electrical conduit 61 comprises one or more wires that are electrically connected to exposed proximal portion 73 (such as by soldering or with a conductive adhesive) and which are connectable to a source of stimulating energy. In certain examples, electrical conduit 61 includes a DIN connector on the end opposite the end connected to conductive shaft 59 to allow for connection of nerve identification device 40 a wide range of known surgeon interface modules 22.

The connection between electrical conduit 61 and conductive shaft proximal exposed portion 73 provides an electrically conductive path that extends through lumens 72, 67, and 65. Because only the exterior portion of exteriorly insulated portion 76 is insulated, the electrically conductive path further extends from exposed proximal portion 73 to nerve stimulating electrode 55. Thus, stimulation energy can be supplied to a patient by contacting nerve stimulating electrode 55 to the patient's skin and supplying stimulation energy to electrical conduit 61. In certain examples, electrical conduit 61 receives nerve stimulation energy from electrical stimulation generator 34.

As mentioned previously, nerve identification device 40 also includes a skin marker 47 (FIG. 2F). In the example of FIGS. 2A-2H, skin marker 47 extends through the lumen 77 of conductive shaft 59. In preferred embodiments, nerve identification device 40 is a single, handheld unit that includes both stimulating electrode 55 and skin marker 47. In certain preferred examples, skin marker 47 includes a skin marking tip 56 that is spaced apart distally from handpiece 42 along the direction of device length L. At least a portion of skin marker 47 is selectively extendable in a direction away from handpiece 42. Skin marker 47 further comprises a proximal end 53 attached to a user control 66 (FIGS. 2B, 2F). User control 66 may comprise a variety of manually manipulable control devices such as buttons, knobs, levers, or sliders. In the depicted example of FIGS. 2A-2H, user control 66 is a slider that is manipulable along a portion of the length of handpiece 42.

Skin marking tip 56 is preferably non-conductive. Skin marking tip 56 is also preferably electrically insulated from conductive shaft 59. Suitable skin marking tip 56 materials include felt and porous ceramics capable of delivering skin marking ink to a patient's skin. In certain examples, skin marker 47 includes a skin marking shaft 49 that is generally rigid to facilitate the deployment of skin marking tip 56. Skin marker 47 also includes plastically deformable section 51 that is distally adjacent to skin marking shaft 49. In one example, skin marking shaft 49 comprises a medical grade stainless steel, and plastically deformable section 51 comprises a plastic that electrically insulates skin marking tip 56 from stimulating energy supplied to conductive shaft 59.

Skin marker 47 includes a lumen 43 (not shown) in which skin marking ink is located. Lumen 43 is preferably in fluid communication with skin marking tip 56 to allow skin marking ink to saturate skin marking tip 56, allowing the ink to be applied from the tip 56 to a patient's skin. As ink in the skin marking tip 56 is consumed, it is replaced by ink flowing from within lumen 43 to tip 56. In this configuration, the skin marking ink flow path is parallel to the path of nerve stimulating energy and is along the longitudinal axis of conductive shaft 59.

A first portion of skin marker 47 remains slidably disposed within lumen 77 of conductive shaft 59, and a second portion of skin marker 47 remains slidably disposed within handpiece 42 but projects proximally away from conductive shaft lumen 77. Nerve stimulating electrode 55 includes a distal opening 57 through which skin marking tip 56 can be selectively extended and retracted. Nerve stimulating electrode 55 also includes a proximal opening (not shown) on its end opposite distal opening 57 for receiving skin marking tip 56. Thus, when a user urges control 66 toward nerve stimulating electrode 55, proximal end 53 of skin marker 47 moves distally within handpiece 42, a portion of skin marker 47 slides distally within lumen 77, and skin marking tip 56 (which is spaced apart from handpiece 42) extends away from nerve stimulating electrode 55. When a user urges control 66 toward proximal handpiece end 46, proximal end 53 of skin marker 47 slides proximally along the length direction L of handpiece 42, and skin marking tip 56 retracts within nerve stimulating electrode 55, or depending on the design, to a position that is proximally spaced apart from nerve stimulating electrode 55. In FIGS. 2A and 2B, skin marking tip 56 is deployed, and a minimum length of skin marking shaft 49 projects proximally away from proximal end 54 of conductive shaft 59. FIG. 2H shows the distal end 52 of conductive shaft 59 with skin marking tip 56 in a refracted condition, such that it is not visible. In this configuration, a maximum length of skin marking shaft 49 projects proximally away from proximal end 54 of conductive shaft 59. FIG. 2G shows distal end 52 of conductive shaft 59 with skin marking tip 56 in an extended condition in which it projects through electrode distal opening 57 and away from electrode 55. A portion of plastically deformable section 51 of skin marker 47 also projects away from electrode 55. In FIGS. 2A and 2B, skin marking tip 56 is also in an extended condition projecting away from electrode 55. This configuration allows a user to extend the skin marking tip 56 away from nerve stimulating electrode 55 without moving the position of nerve stimulating electrode 55 on the surface of the patient's skin because the marking area is within the outer area defined by the nerve stimulating electrode distal facing face 79 which contacts the patient's skin. Nerve identification device 40 may be gripped in a single hand and held therein while supplying nerve stimulating energy to the patient's skin and while marking the skin with skin marking ink.

Nerve stimulating electrode 55 may be provided in a variety of outer surface shapes, such as cylindrical, spherical (also known as “ball-tip”), and frustoconical. However, in each case, a lumen is preferably provided through the electrode to provide a pathway for movement of skin marking tip 56. The distal facing face 79 of electrode 55 may be flush (substantially planar) or may be semi-spherical. In the example of FIGS. 2G and 2H, electrode 55 is annular and includes a proximal cylindrical surface portion 78a and a distal frustoconical surface portion 78b having a flush face 79 (FIG. 2H). Opening 57 is provided within the flush face 79.

When nerve stimulating energy is supplied from nerve identification device 40 to a patient's nerves, a return electrical path is typically provided wherein the stimulating current flows from a supply electrode through the patient and to a return electrode connected to ground. Electrode 55 may be a monopolar or bipolar electrode. A mono polar electrode is a single electrode, whereas a bipolar electrode is effectively two electrodes or two electrode regions that are electrically isolated from one another. Bipolar electrodes may be concentric, or they may be spaced apart from one another. In the case of a concentric bipolar electrode, electrode 55 may comprise two coaxial electrode regions, one from which stimulating energy is supplied to the patient (supply nerve stimulating electrode) and one to which stimulating energy returns from the patient (return nerve stimulating electrode). Alternatively, device 40 may include separate spaced apart electrodes, one of which is used for the supply path of stimulating energy (supply nerve stimulating electrode), and the other of which is used as the return path for stimulating energy (return nerve stimulating electrode). In the case of a monopolar electrode, a separate return electrode is typically attached to the patient to provide a return electrical path.

Suitable nerve stimulating electrodes that are commercially available include off the shelf EMG surface electrodes, such as the Medtronic ENT monopolar (flush tip and ball tip) and bipolar (concentric and side-by-side) stimulating electrodes, and NuVasive Spine's Neurovision EMG electrodes. Although percutaneous electrodes (e.g., needle electrodes) can be used, in certain preferred embodiments, nerve stimulating electrode 55 is designed for contacting the surface of the skin without puncturing the skin and without percutaneous insertion of the electrode.

Handpiece 42 middle section 60 includes a window 50 within which user control 66 is retained. Window 50 has a length along the length of handpiece 42 and along which user control 66 may be moved by a user. User control 66 and/or handpiece 42 may be configured to limit the travel of user control 66 along the length direction L of handpiece 42, and correspondingly, to limit the travel of skin marker 47 relative to handpiece 42, conductive shaft 59, and nerve stimulating electrode 55 in the length direction of device length L. In one example, user control 66 abuttingly engages the handpiece 42 at the ends of window 50 to limit the travel of user control 66 and skin marker 47. As user control 66 is urged in the proximal direction, its proximal end face 63a eventually comes into abutting engagement with the proximal end 68a of window 50 (see FIG. 2B), thereby limiting the extent of retraction of skin marking tip 56 within nerve stimulating electrode 55 or conductive shaft 59. As user control 66 is urged in the distal direction, its distal face 63b eventually comes into abutting engagement with distal end 68b of window 50 (see FIG. 2B), thereby limiting the extent of extension of skin marking tip 56 away from conductive shaft 59 or away from nerve stimulating electrode 55. In the example of FIGS. 2A-2H, user control 66 has a thickness such that a portion of control 66 is located radially inward of window 50 and a portion of control 66 is located radially outward of window 50. The radially inward located portion is connected to proximal end 53 of skin marking shaft 49. Because of this positioning, control 66 engages the ends 68a and 68b of window 50 to limit the travel of user control 66 and skin marking shaft 49.

Other types of mechanical limiting devices may be used to limit the travel of user control 66 and skin marker 47 with respect to handpiece 42. For example, control knob 66 may be spaced apart from window 50 and may have a radial post that extends into window 50 and connects to proximal end 53 of skin marking shaft 49 so that the abutting engagement of the radial post with window ends 68a and 68b limits the travel of the user control 66 and skin marker 47. In addition, skin marker shaft 49 may be spring loaded such that user control 66 releases compressed spring energy to cause skin marker 47 to deploy distally.

Nerve identification device 40 may be provided in a system for identifying nerve locations. In one example, nerve identification device 40 is provided with the surgeon interface module 22 of FIG. 1 and connected thereto to receive nerve stimulation energy from electrical stimulation generator 34. Nerve response sensor electrode 25 is also provided to sense evoked nerve responses generated by stimulated nerves. The nerve response sensor electrode 25 is electrically connected to evoked nerve response monitoring unit 28 (via a wired connection or a wireless signal transmission) so that sensed evoked nerve response data may be stored in data storage device 32 and processed by processor 30. In certain exemplary implementations, an executable computer program is stored in storage device 32 for execution by processor 30 to receive sensed evoked nerve response data and determine if it is indicative of a compound action potential. In certain examples, the program compares sensed evoked nerve response data to baseline nerve response data known to be indicative of compound action potentials to determine whether a compound action potential has been evoked.

Nerve identification device 40 and electrical stimulation generator 34 are preferably configured to deliver sufficient stimulating energy to the surface of a patient's skin to stimulate nerves lying beneath the skin. In certain embodiments, nerve identification device 40 is designed to transmit nerve stimulating currents of at least about 30 mA, more preferably at least about 50 mA, and still more preferably of at least about 100 mA to a patient's skin, and electrical stimulation generator 34 is designed to generate such currents and transmit them to electrical conductor 61. Electrical stimulation generator 34 is also preferably configured to deliver stimulation energy at frequencies of from about 1 Hz to about 50 Hz. In certain examples, electrical stimulation generator 34 delivers single phase or biphasic stimulation energy.

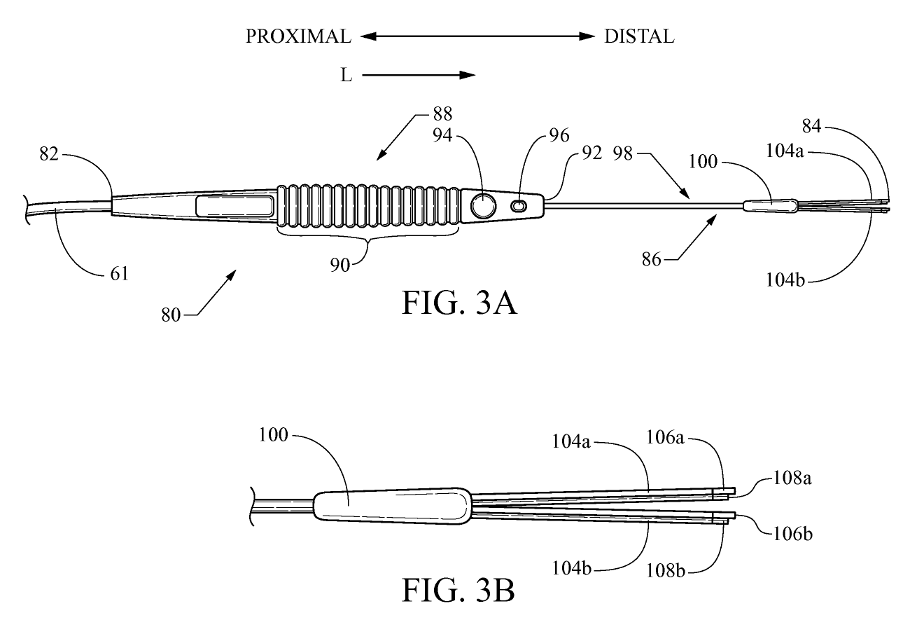

Other nerve identification devices comprising a nerve stimulating electrode and a skin marker may be provided in accordance with the present disclosure. Referring to FIGS. 3A and 3B, nerve identification device 80 is depicted. Nerve identification device 80 is bipolar. Nerve identification device 80 includes a handpiece 88 and shaft section 86. Shaft section 86 includes a proximal portion 98, a hub 100, a first distal shaft portion 104a, and a second distal shaft portion 104b. First distal shaft portion 104a includes a stimulating supply electrode 106a and a first skin marker tip 108a (FIG. 3B). Second distal shaft portion 104b includes a stimulating return electrode 106b and a second skin marker tip 108b. Stimulating supply electrode 106a and stimulating return electrode 106b are spaced apart from one another in a direction perpendicular to the length direction L of nerve identification device 80. First distal shaft portion 104a and second distal shaft portion 104b form a “V” shape when viewed from one or more directions perpendicular to the direction of device length L. Each nerve stimulating electrode 106a and 106b is connected to a respective conductive shaft (not shown) that is disposed within a lumen of its corresponding distal shaft portion 104a and 104b and which is connected to the distal side of hub 100. In certain examples, each skin marking tip 108a and 108b is similarly connected to a marking shaft (not shown) that is also disposed within the lumen of corresponding distal shaft portion 104a and 104b and through which skin marking ink is provided to the skin marking tips 108a and 108b.

As shown in FIGS. 3A and 3B, proximal shaft portion 98 connects hub 100 to handpiece distal end 92. Proximal shaft portion 98 is preferably a non-conductive catheter, and preferably includes a lumen through which separate conductive shafts (not shown), one for each of the electrodes 106a and 106b, is routed. The separate conductive shafts are preferably exteriorly insulated from one another to prevent the formation of a short circuit between them. The shafts connected to electrodes 106a and 106b terminate within handpiece 88 and are connected to corresponding electrical conductors 61 that enter handpiece 88 at conductor port 82.

In certain examples, skin marking tips 108a and 108b are preferably connected to corresponding skin ink shafts (not shown) that each include a lumen through which skin marking ink is supplied to corresponding tips 108a and 108b. The skin ink shafts (not shown) may join in hub 100 to define a single skin ink shaft (not shown) that is located between hub 100 and distal handpiece end 92 and which is disposed in the lumen of shaft 98. Thus, within shaft 98, the skin marker is parallel to, and not coaxial with, conductive shafts that connect to the bipolar nerve stimulating electrodes 106a and 106b. In contrast, the skin marker 47 and conductive shaft 59 of nerve identification device 40 shown in FIGS. 2A-2H are coaxial. Like nerve identification device 40, nerve identification device 80 can be used to apply ink to a patient's skin without moving the nerve stimulating electrodes 106a and 106b on the patient's skin. Nerve identification device 80 can also be gripped in a single hand for supplying stimulating energy to the skin and subsequently marking the stimulated location.

Handpiece 88 includes tactile features, such as ridges 90, to facilitate gripping by a surgeon. Handpiece 88 also includes an ink activation button 94 that is operatively connected to a source of skin marking ink within nerve identification device 80 and which causes the ink to be supplied to skin marking tips 108a and 108b when depressed. In one example, ink is supplied to both tips 108a and 108b from a single ink reservoir upon depressing ink activation button 94. In another example, each tip 108a and 108b receives ink from its own corresponding ink reservoir. In a further example, tips 108a and 108b remain disposed within their own corresponding ink reservoirs in an undeployed condition and are then extended in the distal direction away from hub 100 when deployed by depressing ink activation button 94. For example, each tip 108a and 108b could be operatively connected to a corresponding spring loaded shaft that is deployed upon depressing ink activation button 94 such that when the springs are compressed, the tips 108a and 108b are undeployed and held within their reservoirs.

As with nerve identification device 40, nerve identification device 80 may be supplied with a system for identifying nerve locations. In one example, nerve identification device 80 is provided with the surgeon interface module 22 of FIG. 1 and connected thereto to receive nerve stimulation energy from electrical stimulation generator 34. Nerve response sensor electrode 25 (FIG. 1) is also provided to sense evoked nerve responses generated by stimulated nerves in the manner described previously.

Nerve identification device 80 may also receive feedback from surgeon interface module 22 to indicate to a user when a specified evoked nerve response has been sensed, such as a compound action potential. Visual indicator light 96 is operatively connected to surgeon interface module 22 via one of several conductive leads comprising part of electrical conductor 61. When a specified evoked nerve response is detected (such as by the use of a program stored in storage device 32 and executed by processor 30), an electrical signal is transmitted to visual indicator light 96 to illuminate it, thereby providing a visual indication to the surgeon that a stimulated nerve has evoked a selected response, such as a compound action potential. Such a visual indicator light may also be provided on handpiece 42 of nerve identification device 40, although one is not shown in FIGS. 2A-2H. Surgeon interface module 22 may also be programmed to generate audible signals or alarms when a specified nerve response—such as a compound action potential—is evoked.

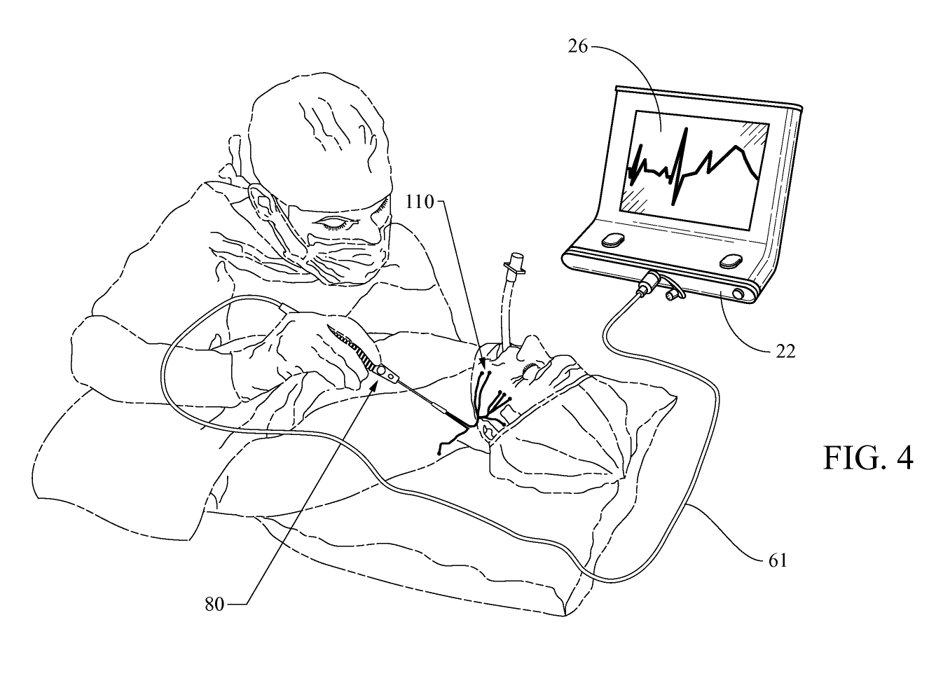

A method of using a nerve identification device such as devices 40 or 80 to identify a nerve location will now be described with reference to FIG. 4. In FIG. 4, nerve identification device 80 is depicted. However, nerve identification device 40 may also be used. Electrical conduit 61 is connected to a surgeon interface module 22 that includes a monitor 26. Although not shown, a foot switch may also be connected to surgeon interface module 22 to allow the surgeon to selectively supply and/or modulate the level of stimulation energy using his or her foot.

As mentioned previously, nerve identification device 80 is bipolar and includes both supply (106a) and return (106b) electrodes for supplying stimulating energy to the surface of a patient's skin. A nerve response sensor electrode 25 (FIG. 1) is also placed on the patient's skin to sense evoked nerve responses.

As shown in FIG. 4, the surgeon grasps the nerve identification device 80 in a single hand and contacts regions on the skin (in this case, the face, neck and shoulder areas) with nerve stimulating electrodes 106a and 106b. Nerve stimulating electrodes 106a and 106b contact the skin but do not puncture it and are not otherwise inserted percutaneously. Using a footswitch or another control, the surgeon selects a desired level of nerve stimulating energy and supplies the energy to the locations of the patient's skin which are in contact with nerve stimulating electrodes 106a and 106b. The nerve response sensor electrode 25 will then detect electrical activity and feed corresponding electrical signals to surgeon interface module 22 for processing by processor 30 and storage by storage unit 32 (FIG. 1).

The evoked nerve responses are displayed on monitor 26. Processor 30 executes a program that compares data supplied by the nerve response sensor electrode 25 to baseline evoked nerve response data to determine if a compound action potential has been detected. If one has been detected, the surgeon is alerted by audible or visual signals on the surgeon interface module and/or the nerve identification device 80 (e.g., by using visual indicator 96 shown in FIG. 3A). The surgeon then depresses ink activation button 94 while holding the skin marking tips 108a and 108b in the same location at which the stimulating energy was supplied and marks the location to indicate that nerves are located in the area. If nerve identification device 40 were instead used, the surgeon would urge user control 66 in the distal direction, thereby causing the skin marking tip 56 to project outwardly from electrode 55 and through electrode opening 57 (FIG. 2F) and would then mark the area accordingly. The application of skin marking ink with either device 40 or 80 is preferably done while holding the device 40 or 80 in the same single hand used when nerve stimulating energy is supplied to the patient, thereby improving the accuracy of the ink placement relative to the location to which the CAP-evoking stimulating energy is supplied.

In certain examples, the baseline signals to which evoked nerve responses are compared are simply the stimulating energy signals. The supplying of stimulation energy may itself cause electrical activity to be detected by the evoked nerve response sensor electrode 25 even if the activity was not caused by nerve stimulation.

Nerve stimulation energy may be supplied in a variety of different ways. In one example, a selected stimulation energy level (e.g., 50 mA) is used. However, certain CAPs are only evoked in response to a particular threshold stimulating energy. Thus, in another example, the stimulation energy level is progressively increased until the evoked response shows no further increase, and the maximum evoked response is analyzed to determine if it is indicative of a compound action potential. In one variant of this technique, the stimulation energy level is increased by a selected amount after the maximum evoked response is observed to confirm that no further increases in the maximum evoked response occur. The level of stimulation energy beyond that which evokes a maximum response is sometimes referred to as a “supramaximal” stimulating energy level.

The level of stimulating energy provided by electrical stimulation generator 34 may be manually increased by the surgeon or a surgeon's assistant progressive manipulation of a user control (e.g., a footswitch, knob, etc.) to increase the output of the electrical stimulation generator 34. However, in one preferred implementation, processor 30 of surgeon interface module 22 includes an executable program that interfaces with an electrical stimulation generator controller (not shown) and performs an automatic calibration procedure to determine the supramaximal stimulating energy level at each location of interest proximate to a targeted motor nerve.

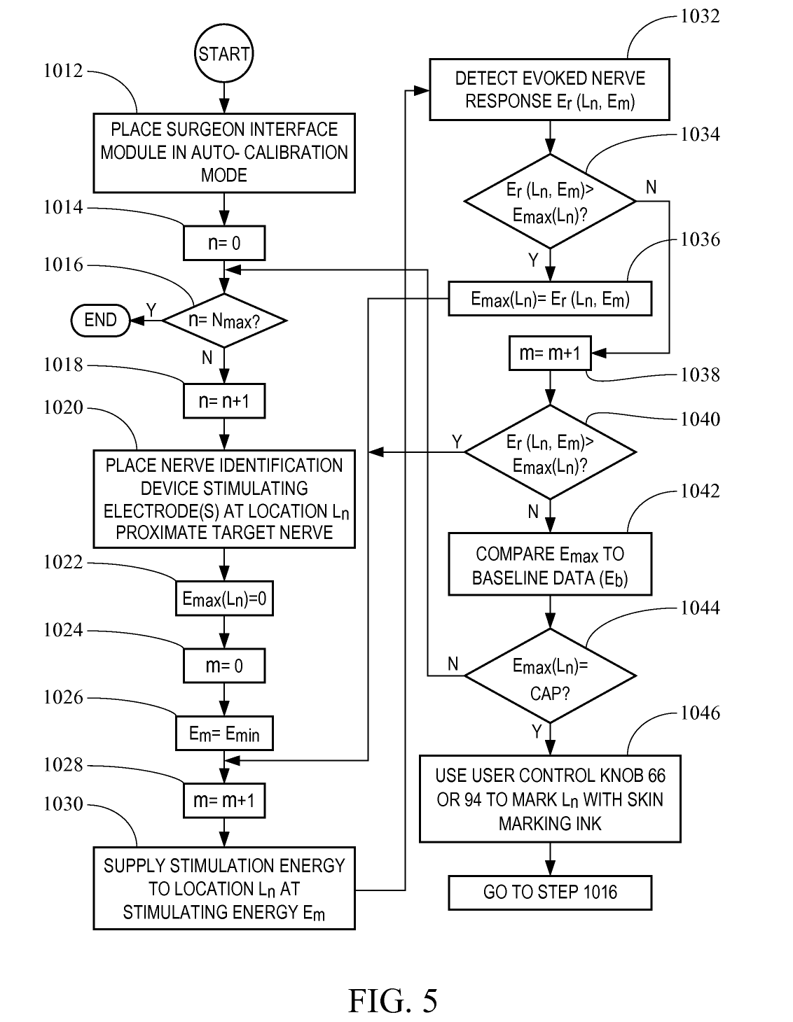

Referring to FIG. 5, a method of identifying a nerve location that includes automatic calibration of the stimulation energy delivered by nerve identification devices 40, 80 is described. In accordance with the method, in step 1012 the surgeon interface module 22 is first placed in an auto-calibration mode such that a calibration program resident in processor 30 executes to progressively increase the stimulation energy provided by electrical stimulation generator 34 to stimulating electrodes 55 or 106a/106b. The surgeon interface module 22 may be placed in auto-calibration mode using standard user interface controls, such as a knob, lever, icons, or using a touch screen.

In FIG. 5, the variable “n” is a location index which corresponds to a unique location on a patient's skin proximate the motor nerve of interest. In step 1014, the location index n is initialized to zero. In step 1016, the location index is compared to the maximum location index Nₘₐₓ, which represents the index of the last location to be stimulated. If the maximum location index Nₘₐₓ has been reached, the automatic calibration program terminates. Nₘₐₓ will not necessarily be a programmed variable, and in certain embodiments, the surgeon may carry out step 1016 manually by stimulating a number of locations and then deciding to stop. In that case, Nₘₐₓ will not have a preset or selected value, but rather, will simply reflect the fact that after stimulating some number of locations, the surgeon stops.

In step 1018, the location index n is incremented by one. The nerve identification device 40, 80 is then placed such that its stimulating electrode(s) 55, 106a/106b contact a location on the patient's skin Lₙ which corresponds to the current value of the location index n. Step 1020. In step 1022, the maximum evoked nerve response signal value Eₘₐₓ(Lₙ) for the current location Lₙ is initialized to zero. In step 1024, the stimulation energy level index, m, is initialized to zero. The current value of the stimulating energy level, Eₘ, is initialized to a minimum desired stimulation level, Eₘᵢₙ. Step 1026. Although not separately shown, following step 1026, the minimum stimulation energy Eₘᵢₙ may be supplied to location Ln, after which control transfers to step 1032. In a modified version of the method of FIG. 5, step 1026 is omitted and the value of E at m=1 (i.e., E(1)) is simply set to Eₘᵢₙ after step 1028.

In step 1028, the stimulating energy level index m is incremented by one. In step 1030 nerve stimulation energy is supplied from electrical stimulation generator 34 to stimulating electrodes 55, 106a/106b at the stimulation energy level Eₘ corresponding to the current value of the index m. In step 1032, a nerve response sensor electrode 25 (FIG. 1) placed on the patient's skin detects an evoked nerve response signal Eᵣ (Lₙ, Eₘ) that corresponds to the current location Lₙ and stimulation energy level Eₘ. The signal may or may not actually be a nerve response signal but is denoted as such herein because it is detected by a nerve response sensor electrode. In some cases, the detected signal may simply be indicative of the stimulating energy level, such as when the stimulation energy does not reach a nerve.

In step 1034, the current evoked nerve response signal Eᵣ (Lₙ, Eₘ) is compared to the maximum value of the evoked nerve response signal Eₘₐₓ(Lₙ) at the current location Lₙ of the stimulation electrodes 55, 106a/106b on the patient's skin. The maximum value Eₘₐₓ (Lₙ) is retrieved from storage device 32 to make the comparison. If Eᵣ (Lₙ, Eₘ) exceeds the maximum evoked nerve response signal Eₘₐₓ(Lₙ), then Eᵣ (Lₙ, Eₘ) becomes the new maximum value Eₘₐₓ(Lₙ) (Step 1036) and control returns to step 1028. Although not shown in FIG. 5, in certain preferred implementations, a maximum nerve stimulation energy level is selected, and once it is reached control returns to step 1016. This prevents the patient from being subjected to excessive stimulation energy levels that could cause tissue damage.

When Eᵣ(Lₙ, Eₘ) is not greater than the maximum value Eₘₐₓ (Lₙ), it is possible that any further increases in the stimulating energy Eₘ will not produce an increase in the evoked nerve response signal Eᵣ (Lₙ, Eₘ), which means that Eₘ is at or above the maximum-response generating nerve stimulation energy level. In certain exemplary implementations, the evoked nerve response signal Eᵣ (Lₙ, Eₘ) is then evaluated in step 1042 (such as by comparison to a baseline signal E_{b}, with or without signal filtering) to determine if the evoked nerve response signal Eᵣ (Lₙ, Eₘ) is indicative of a compound action potential (step 1044), in which case the skin is marked by deploying skin marker 47 using user control 66 (for nerve identification device 40) at the location Lₙ to indicate that a nerve is present, or in the case of nerve identification device 80 by activating button 94 to cause ink to flow to marker tips 108a/108b. Step 1046. The process may then return to step 1016 to stimulate another location Lₙ on the patient's skin (if the final location, Nmax has not been reached, in which case the method ends). However, in the method of FIG. 5, a “supramaximal” response is generated to confirm that Eᵣ (Lₙ, Eₘ) is actually the maximum observable nerve response. Thus, following step 1034, the stimulating energy level index m is incremented by one (step 1038) and stimulation energy is supplied (not separately shown in FIG. 5) to location L. If the evoked nerve response signal Eᵣ (Lₙ, Eₘ) is greater than the stored maximum value Eₘₐₓ (Lₙ) in step 1040, the maximum observable response was not observed in step 1034, and the process returns to step 1028 (unless the maximum stimulation energy has been reached, in which case control returns to step 1016 even though no comparison of the stimulation energy to the maximum stimulation energy is shown in FIG. 5). If, instead, the evoked nerve response signal Eᵣ (Lₙ, Eₘ) in step 1040 is not greater than the maximum value Eₘₐₓ (Lₙ), then the level of stimulation energy is “supramaximal” and the maximum evoked nerve response signal Eₘₐₓ (Lₙ) is compared to baseline evoked nerve response data E_{b} (Step 1042) to determine if a compound action potential (CAP) has been evoked. Step 1044. If a CAP has been evoked, the surgeon deploys the nerve identification device skin marker by using user control 66, 94 and marks the skin at location Lₙ to indicate the presence of a nerve. Step 1046. If a CAP has not been generated, the method returns to step 1016. If the final location has been reached, the location index n will equal the maximum location index Nₘₐₓ, and the method terminates. Otherwise, control returns to step 1018 so that the next location Lₙ can be stimulated.

The stimulating energy level increments (ΔEₘ=Eₘ−Eₘ₋₁) may be held constant during the method of FIG. 5, or they may vary. The increments may also be tuned to the sensitivity of the evoked nerve responses. In certain examples, ΔEₘ is at least about 0.5 mA, preferably at least about 1 mA, and more preferably at least about 2 mA. In other examples, ΔEₘ is no more than about 10 mA, preferably no more than about 8 mA, and more preferably no more than about 6 mA. In one example, ΔEₘ is about 5 mA.

The foregoing methods of identifying nerve locations using nerve identification devices such as devices 40, 80 can be used in a variety of applications, including the destruction of degenerate nerves, pain treatment, the treatment of obstructive sleep apnea, and the performance of surgical procedures.

Following the use of the methods of nerve identification described herein, the patient's skin will be marked with skin ink to indicate the location of nerves. In one application, the methods of nerve identification are used to identify and non-invasively destroy degenerate nerves. Once the nerve locations are identified and marked, the stimulating electrodes 55, 106a/106b of nerve identification devices 40, 80 are placed in contact with the marked locations on the patient's skin. Electrical stimulation generator 34 is then activated to supply nerve destroying energy to the marked locations. In certain examples, a minimum nerve destroying energy is applied, and the nerve destroying energy is then increased in increments until a maximum nerve destroying energy is reached. Exemplary minimum nerve destroying energies are at least about 60 mA, preferably at least about 65 mA, and more preferably at least about 70 mA. Exemplary maximum nerve destroying energies are preferably no more than 140 mA, more preferably no more than 130 mA, and even more preferably no more than about 120 mA. Preferred nerve destroying energy increments are preferably at least about 1 mA, more preferably at least about 3 mA, and even more preferably at least about 5 mA. Preferred nerve destroying energy increments are preferably no more than about 15 mA, more preferably no more than about 12 mA, and even more preferably no more than about 10 mA. The nerve destroying energy levels are preferably sufficient to destroy the targeted degenerate nerves indicated by the skin markings without causing significant damage to neighboring tissues and structures (e.g., blood vessels). In a variation on this method, after identifying the locations on the skin where compound action potentials are generated, percutaneous electrode needles may be inserted at the skin location markings and used to supply nerve destroying energy.

The nerve identification methods described herein can also be used in conjunction with pain treatment methods. In one method of treating pain caused by a region of nerves at a nerve location, the methods of nerve identification are used to identify the location of nerves that are causing the patient pain. The stimulating electrodes 55, 106a/106b of nerve identification devices 40, 80 are placed in contact with the marked skin areas, and electrical stimulation generator 34 is activated to supply pain relief energy to the marked areas. Preferred pain relief energy levels may vary depending on the particular indication. For example, in lower lumbar applications, the maximum pain relief energy is preferably no more than about 20 mA, even more preferably no more than about 17 mA, and even more preferably no more than about 15 mA. In other pain applications or for mood alteration applications, maximum pain relief energy levels are preferably no more than about 2 mA, more preferably no more than about 3 mA, and even more preferably no more than about 4 mA. In a variation on this method, after identifying the locations on the skin where compound action potentials are generated, percutaneous electrode needles may be inserted at the skin location markings and used to supply pain relief energy.

In yet another application, a method of treating sleep apnea is performed by first performing the methods of nerve identification described herein to identify the hypoglossal nerve. The hypoglossal nerve innervates the genioglossus muscle, which in certain patients, causes the tongue to move so as to open a portion of the airway to reduce the occurrence of sleep apnea. In accordance with the method, the methods of nerve identification described herein are carried out on a patient's neck and/or face to locate and mark areas on the patient's skin proximate the hypoglossal nerve. The stimulation electrodes 55, 106a/106b are placed in contact with the marked skin locations, and genioglossus innervating stimulation energy levels are supplied by electrical stimulation generator 34. In one preferred implementation, biphasic genioglossus innervating stimulation energy is supplied. In certain preferred examples, the stimulation of marked locations occurs while the patient is asleep, in which case the biphasic innervating stimulation energy may be supplied by nerve stimulating electrodes that are adhesively attached to the marked locations on the skin that are indicative of the hypoglossal nerve. In certain examples, biphasic innervating stimulation energy is supplied at a minimum level and incremented until a maximum level is reached. Exemplary minimum biphasic innervating stimulation energies are at least about 60 mA, preferably at least about 65 mA, and more preferably at least about 70 mA. Exemplary maximum biphasic innervating stimulation energies are preferably no more than 120 mA, more preferably no more than 115 mA, and even more preferably no more than about 110 mA. In a variation on this method, after identifying the locations on the skin where compound action potentials are generated, percutaneous electrode needles may be inserted at the skin location markings and used to supply biphasic innervating stimulation energy.

In still another application, nerve identification devices such as devices 40, 80 can be used in preoperative nerve identification procedures. In accordance with one such method, a proposed incision region is identified on the patient's skin. Stimulation electrodes 55, 106a/106b are placed in contact with locations within the region, and nerve stimulating energy is supplied to the locations. Evoked nerve responses are then detected by an evoked nerve response sensor electrode 25 placed on the patient's skin. It is then determined whether the evoked nerve response is indicative of a nerve, such as by determining whether the evoked nerve response shows a compound action potential. If a compound action potential is indicated, user control 66, 94 is then activated to mark the skin with skin ink. The procedure is repeated at a number of locations within the incision region. An incision is then made in the incision region, but away from the skin marking to preserve the integrity of the nerve(s) identified by the markings In one example, the method of FIG. 5 is carried out to identify the location of nerves in the incision region using autocalibrated, supramaximal nerve stimulation energy levels.

The present invention has been described with reference to certain exemplary embodiments thereof However, it will be readily apparent to those skilled in the art that it is possible to embody the invention in specific forms other than those of the exemplary embodiments described above. This may be done without departing from the spirit of the invention. The exemplary embodiments are merely illustrative and should not be considered restrictive in any way. The scope of the invention is defined by the appended claims and their equivalents, rather than by the preceding description.

What is claimed is:

1. A nerve identification device, comprising:
a handpiece;
a nerve stimulating electrode; and
a skin marker.

2. The nerve identification device of claim 1, wherein the skin marker is in fluid communication with a source of skin marking ink.

3. The nerve identification device of claim 1, further comprising a shaft connected to the handpiece, wherein the stimulating electrode is connected to the shaft.

4. The nerve identification device of claim 1, wherein the skin marker comprises a skin marking tip that is spaced apart from the handpiece and selectively extendable in a direction away from the handpiece.

5. The nerve identification device of claim 4, further comprising a user control connected to the handpiece, wherein moving the user control causes the skin marking tip to move relative to the stimulating electrode.

6. The nerve identification device of claim 1, wherein the skin marker comprises a skin marking tip that is selectively extendable away the stimulating electrode.

7. The nerve identification device of claim 1, wherein the skin marker comprises a shaft that is moveable relative to the handpiece, the skin marker shaft having a proximal end disposed within the handpiece and a distal end spaced apart from the handpiece.

8. The nerve identification device of claim 1, wherein the skin marker comprises a skin marking tip that is electrically insulated from the skin marking electrode.

9. The nerve identification device of claim 1, wherein the nerve stimulating electrode includes an opening, and the skin marker has a tip that is selectively extendable through the nerve stimulating electrode opening.

10. The nerve identification device of claim 1, comprising one or more conductive leads, each having a first end connectable to a source of nerve stimulation energy and a second end disposed in the handpiece and in electrical communication with the stimulation electrode.

11. The nerve identification device of claim 1, wherein the nerve stimulating electrode is annular.

12. The nerve identification device of claim 1, wherein the nerve stimulating electrode is a first nerve stimulating electrode, and the device further comprises a second nerve stimulating electrode spaced apart from the first nerve stimulating electrode.

13. The nerve identification device of claim 1, wherein the nerve stimulating electrode is a first nerve stimulating electrode, the device further comprises a second nerve stimulating electrode, and the first and second nerve stimulating electrodes are coaxial.

14. The nerve identification device of claim 1, wherein the nerve stimulating electrode has a tip with a substantially flat distal surface.

15. The nerve identification device of claim 1, wherein the nerve stimulating electrode has a substantially spherical tip.

16. The nerve identification device of claim 1, wherein the nerve stimulating electrode is stationary relative to the handpiece.

17. The nerve identification device of claim 1, further comprising a nerve response indicator located on the handpiece.

18. A system for identifying nerve locations, comprising:
the nerve identification device of claim 1;
a nerve response sensor electrode;
an electrical stimulation generator in electrical communication with the nerve stimulation electrode; and
an evoked nerve response monitoring unit in electrical communication with the nerve response sensor electrode.

19. The system of claim 18, wherein the evoked nerve response monitoring unit includes a CPU and a storage device, and the storage device includes a program for detecting compound action potentials based on an evoked nerve response received from the nerve response sensor electrode.

20. The system of claim 18, further comprising a foot switch that is actuatable to cause electrical stimulation energy to be supplied from the electrical stimulation generator to the nerve stimulation electrode.

21. The system of claim 18, wherein the electrical stimulation generator supplies nerve stimulation current of at least about 100 mA.

22. A method of identifying a nerve location using the nerve identification device of claim 1, comprising:
contacting a location on a patient's skin with the stimulation electrode;
supplying stimulation energy to the stimulation electrode;
detecting an evoked nerve response;
determining whether the evoked nerve response is indicative of a motor nerve; and
marking the location on the patient's skin with the skin marker if the evoked nerve response is indicative of a motor nerve.

23. The method of claim 22, wherein the skin marker comprises a skin marking tip, and the step of marking the skin with the skin marker comprises extending the skin marking tip in a direction away from the stimulation electrode.

24. The method of claim 22, further comprising gripping the handpiece with a single hand, and wherein the steps of contacting a location on the patient's skin and marking the location on the patient's skin with the skin marker if the evoked nerve response is indicative of a motor nerve are carried out while gripping the handpiece in the single hand.

25. The method of claim 22, wherein the step of detecting an evoked nerve response comprises receiving evoked nerve response data, and the step of determining whether the evoked nerve response is indicative of a motor nerve comprises calculating a difference between the received evoked nerve response data and baseline evoked nerve response data.

26. A non-invasive method of destroying a degenerate nerve, comprising:
performing the method of claim 22 to identify a degenerate nerve location on the patient's skin;
contacting the marked location on the patient's skin with the stimulation electrode; and
supplying nerve destroying energy to the stimulation electrode.

27. A method of treating pain caused by a region of nerves located at a nerve location, comprising:
performing the method of claim 22 to identify the nerve location;
contacting the marked location on the patient's skin with the stimulation electrode; and
supplying pain relief energy to the stimulation electrode.

28. A method of treating obstructive sleep apnea, comprising:
performing the method of claim 22 to identify a hypoglossal nerve location on the patient's skin;
contacting the marked location on the patient's skin with the stimulation electrode; and
supplying biphasic stimulation energy pulses to the stimulation electrode to innervate the genioglossus muscle.

29. A method of performing surgery using the nerve identification device of claim 1, comprising:
identifying a proposed incision region on a patient's skin;
contacting at least one location in the proposed incision region with the stimulation electrode;
supplying stimulation energy to the at least one location;
detecting an evoked nerve response;
determining whether the evoked nerve response is indicative of a motor nerve; marking the at least one location on the patient's skin with the skin marker if the evoked nerve response is indicative of a motor nerve; and
making an incision within the incision region away from the marking
